(19) United States
(12) Patent Application Publication
(10) Pub. No.: US 2016/0137969 A1
(43) Pub. Date: May 19, 2016

(54) METHOD FOR STORING EXCESS ENERGY

(71) Applicants: Thomas HAAS, Münster (DE); EVONIK DEGUSSA GMBH, Essen (DE)

(72) Inventor: Thomas HAAS, Muenster (DE)

(73) Assignee: EVONIK DEGUSSA GMBH, Essen (DE)

(21) Appl. No.: 14/898,679

(22) PCT Filed: Jun. 17, 2014
(86) PCT No.: PCT/EP2014/062631

§ 371 Date: Dec. 15, 2015

(30) Foreign Application Priority Data
Jun. 18, 2013 (EP) 13172441.1

(51) Int. Cl. C12M 1/00; F01K 11/02; C12P 7/54; F02C 6/14; C12P 7/42; C12P 7/06

(52) U.S. Cl. CPC C12M 43/04; C12P 7/42; C12P 7/065; C12P 7/54; C12M 29/00; C12M 43/08; F02C 6/14; F01K 11/02

(57) ABSTRACT

The invention relates to a method for utilizing gases containing CO and/or CO₂, which method comprises the following steps: A) providing a gas flow of the gas containing CO and/or CO₂, B) converting at least a portion of the gas flow into electrical energy, C) transforming at least a portion of the gas flow into at least one organic substance using a biotechnological fermentation process, and possibly D) repeating method steps B) and C).

### FIELD OF THE INVENTION

The invention relates to a method for utilizing gases containing CO and/or CO₂ comprising the method steps of:
A) providing a gas stream of gas containing CO and and/or CO₂,
B) converting at least a part of the gas stream into electrical energy,
C) converting at least a part of the gas stream to at least one organic substance in a biotechnological fermentation process and optionally
D) repeating method steps B) and C).

### PRIOR ART

Electricity-producing power plants produce excess electricity during periods of low demand. This has to be stored appropriately in another form.

For instance, pumped storage reservoirs are constructed to store excess electricity. Pumped storage facilities have a large storage capacity but also a large space and area requirement and have a not inconsiderable impact on ecosystems and landscape. Another approach is to store electrical energy in large batteries, particularly lithium ion batteries. However, this technology requires very high investment in additional batteries, the depletion of which nullifies the advantage of using the cheap excess electricity.

Other alternatives are the conversion of electricity into hydrogen which then converts CO₂ chemically to methane. The gas then serves as an energy store. In this case also, considerable investment must be employed in the chemical conversion. In addition, methane has a low energy density and is not readily transportable, since it exists as a gas and requires either a pipeline or an expensive liquefaction. A higher density can be achieved by the chemical conversion into liquids. However, these processes end up either in relatively low value substance mixtures or in methanol, which is also unsuitable as an energy medium due to its high oxygen content.

For power plants that are based on fuels, in addition to the possibility of converting the same to electricity, there exists the option to divert the energy of the fuel prior to generating electricity and to use this energy for generating heat and subsequent heating.

Such a method has been used hitherto, in particular where the fuel is delivered continuously and the delivery cannot be restricted as, for example, in power plants which draw their energy from the waste gas streams of industrial manufacturing plants such as steel works for example.

This process has the disadvantage that heat as energy medium experiences heavy losses due to lack of insulation and accordingly requires prompt use. Heat is likewise poorly transportable such that the heat consumer must be directly located near the power plant.

The object of the present invention is to create the possibility of saving excess energy for power plants based on fuel.

### DESCRIPTION OF THE INVENTION

Surprisingly, it has been found that the method described hereinafter is able to solve the problem posed by the invention.

The present invention thus relates to a method for utilizing gases containing CO and/or CO₂ comprising the method steps of:
A) providing a gas stream of gas containing CO and and/or CO₂,
B) converting at least a part of the gas stream into electrical energy,
C) converting at least a part of the gas stream to at least one organic substance in a biotechnological fermentation process and optionally
D) repeating method steps B) and C).

An advantage of the present invention is that the energy storage of the excess energy takes place prior to the conversion into electricity and thus one less conversion step takes place and thus a higher efficiency can be achieved.

A further advantage of the present invention is that the energy storage of the excess energy may be suspended as required and may therefore over time be carried out discontinuously.

Another advantage of the present invention is that the energy storage of the excess energy does not require any notable start-up time, so large amounts of excess energy can be utilized immediately after the occurrence thereof.

A further advantage of the present invention is that the energy storage of the excess energy has low space and area requirements.

Yet another advantage of the present invention is that the energy storage of the excess energy has a high energy density and thus transport of the energy is greatly facilitated.

A further advantage of the present invention is that the energy storage may be carried out with relatively low investment since no sterile technique is necessary for the fermentation.

Yet another advantage of the present invention is that the energy storage of the excess energy is in liquid form and is thereby easy to transport.

A further advantage of the present invention is that the energy storage is freely scalable in terms of dimensioning.

Yet another advantage of the present invention is that the energy storage can deal with highly fluctuating energy flow and therefore is an ideal buffer.

A preferred method according to the invention is characterized in that method step B) is carried out while method step C) is carried out.

This means that at the same time a part of the gas stream of the gas containing CO and and/or CO₂ is used for generating power, while another part is used for producing the organic substance.

The size of the respective gas streams can be continuously varied and adjusted, preferably to the extent that the amount of excess energy requires.

In extreme cases, even temporarily, the entire gas stream may be used for the production of organic substance, which corresponds to a preferred method according to the invention, which is characterized in that method step B) is not carried out while method step C) is carried out.

On the other hand, even in the case of high power demand, the entire gas stream may be used for conversion to electrical energy, which corresponds to a preferred method according to the invention which is characterized in that method step C) is not carried out while method step B) is carried out.

Method steps B) and C) may be repeated in the method according to the invention, preferably being repeated multiple times, which corresponds to a preferred method according to the invention which is characterized in that method step D) is carried out.

It is advantageous in accordance with the invention if the gas containing CO and/or CO₂ comprises a reducing agent, preferably hydrogen.

This has the technical effect that the required redox equivalents are already incorporated for the biotechnological process in method step C).

The gas containing CO and/or CO₂ is preferably selected from the group of synthesis gas, coke gas, blast furnace gas from blast furnaces, flue gas from the combustion of solid fuels or wastes, gases from a petroleum cracker and volatile substances released during gasification of cellulose-containing materials or coal.

Particularly well suited to the biotechnological conversion, and therefore preferably used in accordance with the invention, is blast furnace gas from a blast furnace in steel-making.

This has the technical effect that method step C) can be operated at high yield since this gas stream has an ideal ratio of CO, CO₂ and hydrogen.

In a preferred alternative embodiment of the method according to the invention, method step A) is characterized in that said method includes the use of incompletely combusted fuel from a coal- or gas-fired power plant.

For this purpose, conventional power plants would have to be converted in such a way that they could burn the fuels to their degree of combustion in a controlled manner, and in the case of excess electricity production the fuel would not be burnt completely to CO₂ but be converted partially to synthesis gas which is then used for method step C).

In a preferred method according to the invention, method step B) includes generating electricity by means of a gas turbine and/or steam turbine process.

A preferred method according to the invention is characterized in that the organic substance in method step C) is selected from organic substances comprising at least three, particularly at least four carbon atoms, preferably 3 to 26, particularly 4 to 20 carbon atoms, which are liquid particularly at 25° C. and 1 bar pressure.

This has the technical advantage that the energy density in the organic substance is high and thus more excess energy is in a readily transportable form.

The organic substance in method step C) is particularly preferably selected from the group of 1-butanol, isobutanol, butanediol, propan-2-ol, acetone, 1-propene, butene, isobutyric acid, 2-hydroxyisobutyric acid, methyl 2-hydroxyisobutyrate, straight-chain and branched alkanoic acids, which may optionally comprise at least one double bond, and derivatives thereof, such as butyric acid, hexanoic acid and esters thereof, and also the corresponding alkanols.

The term “derivatives of alkanoic acids” is understood to mean in particular the reduced forms of alkanoic acid, aldehyde and alcohol, the alkanoic esters, the omega-hydroxylated alkanoic acids, the omega-aminated alkanoic acids, the alkanoic acid amides and the diacids and diamines.

A preferred method according to the invention is characterized in that acetogenic bacteria are used in method step C).

The use of acetogenic bacteria has the technical effect that the gas stream for method step C) can be reduced temporarily to a minimum and may even be completely interrupted. This type of bacteria, that in nature are used to surviving under the most adverse conditions, can remain in the fermenter for a long time without particular care and nutrition. In addition, the use of acetogenic bacteria causes the technical effect that excess energy formed can be utilized immediately since the bacteria, on reinstating the gas stream, immediately restart their metabolism and convert the gas to organic substance.

The term “acetogenic bacterium” is understood to mean a bacterium which is capable of carrying out the Wood-Ljungdahl metabolic pathway and is therefore capable of converting CO and CO₂ and hydrogen to acetate.

The term “acetogenic bacterium” also includes those bacteria which originally as the wild type do not have the Wood-Ljungdahl metabolic pathway but only have this due to genetic modification. Such bacteria may be, for example, E. coli cells.

Acetogenic bacteria used in method step C) preferably have increased enzyme activity of a Wood-Ljungdahl metabolic pathway enzyme owing to genetic modification compared to their wild type. In this context, preferred Wood-Ljungdahl metabolic pathway enzymes are selected from CO dehydrogenases and acetyl-CoA synthetases. Acetogenic bacteria which convert CO₂ and/or CO, and also suitable methods and method conditions which are used in method step C) have been known for a long time.

Such processes are described for example
in WO9800558, WO2000014052, WO2010115054
in Demler et al. Reaction engineering analysis of hydrogenotrophic production of acetic acid by Acetobacterium woodii. Biotechnol Bioeng. 2011 Feb; 108(2): 470-4,
in Younesi et al. Ethanol and acetate production from synthesis gas via fermentation processes using anaerobic bacterium, Clostridium ljungdahlii. Biochemical Engineering Journal, Volume 27, Issue 2, pages 110-119,
in Morinaga et al. The production of acetic acid from carbon dioxide and hydrogen by an anaerobic bacterium. Journal of Biotechnology, Volume 14, Issue 2, pages 187-194,
in Li Production of acetic acid from synthesis gas with mixed acetogenic microorganisms, ISSN 0493644938,
in Schmidt et al. Production of acetic acid from hydrogen and carbon dioxide by clostridium species ATCC 2979. Chemical Engineering Communications, 45:1-6, 61-73,
in Sim et al. Optimization of acetic acid production from synthesis gas by chemolithotrophic bacterium—Clostridium aceticum using a statistical approach. Bioresour Technol. 2008 May; 99(8): 2724-35,
in Vega et al. Study of gaseous substrate fermentations CO conversion to acetate 1 Batch culture and 2 continuous culture. Biotechnology and Bioengineering Volume 34, Issue 6, pages 774 and 785, September 1989,
in Cotter et al. Ethanol and acetate production by Clostridium ljungdahlii and Clostridium autoethanogenum using resting cells. Bioprocess and Biosystems Engineering (2009), 32(3), 369-380 and
in Andreesen et al. Fermentation of glucose, fructose, and xylose by Clostridium thermoaceticum. Effect of metals on growth yield, enzymes, and the synthesis of acetate from carbon dioxide. Journal of Bacteriology (1973), 114(2), 743-51.

The person skilled in the art is offered from this a large number of feasible options for designing method step C) which all function well.

Particular preference is given to using acetogenic bacteria in method step C) selected from the group comprising Clostridium autothenogenum DSMZ 19630, Clostridium ragsdahlei ATCC no. BAA-622, Clostridium autoethanogenum, Moorella sp HUC22-1, Moorella thermoaceticum, Moorella thermoautotrophica, Rumicoccus productus, Acetoanaerobum, Oxobacter pfennigii, Methanosarcina barkeri, Methanosarcina acetivorans, Carboxydothermus, Desulphotomaculum kutznetsovii, Pyrococcus, Peptostreptococcus, Butyribacterium methylotrophicum ATCC 33266, Clostridium formicoaceticum, Clostridium butyricum, Laktobacillus delbrukii, Propionibacterium acidoprprionici, Proprionispera arboris, Anaerobierspirillum succiniproducens, Bacterioides amylophilus, Becterioides ruminicola, Thermoanaerobacter kivui, Acetobacterium woodii, Acetoanaerobium notera, Clostridium aceticum, Butyribacterium methylotrophicum, Moorella thermoacetica, Eubacterium limosum, Peptostreptococcus productus, Clostridium ljungdahlii, Clostridium ATCC 29797 and Clostridium carboxidivorans, in particular ATCC BAA-624. A particularly suitable bacterium is Clostridium carboxidivorans, in particular those strains such as “P7” and “P11”. Such cells are described for example in U.S. 2007/0275447 and U.S. 2008/0057554.

A further particularly suitable bacterium is Clostridium ljungdahlii, in particular strains selected from the group comprising Clostridium ljungdahlii PETC, Clostridium ljungdahlii ERI2, Clostridium ljungdahlii COI and Clostridium ljungdahlii O-52: these are described in WO 98/00558 and WO 00/68407, and also ATCC 49587, ATCC 55988 and ATCC 55989.

In an alternative preferred embodiment of the method according to the invention, ethanol is formed in method step C) and the microorganism used is Alkalibaculum bacchi ATCC BAA-1772, Moorella sp. HUC22-1, Clostridium ljungdahlii, Clostridium ragsdahlei, or Clostridium autoethanogenum. Corresponding instructions for carrying out method step A) can be found for example in Saxena et al. Effect of trace metals on ethanol production from synthesis gas by the ethanologenic acetogen Clostridium ragsdalei. Journal of Industrial Microbiology & Biotechnology Volume 38, Number 4 (2011), 513-521,
Younesi et al. Ethanol and acetate production from synthesis gas via fermentation processes using anaerobic bacterium Clostridium ljungdahlii. Biochemical Engineering Journal Volume 27, Issue 2, 15 December 2005, pages 110-119,
Sakai et al. Ethanol production from H2 and CO2 by a newly isolated thermophilic bacterium, Moorella sp. HUC22-1. Biotechnology Letters Volume 26, Number 20 (2004), 1607-1612 and
Abrini et al. Clostridium autoethanogenum, sp. nov., an anaerobic bacterium that produces ethanol from carbon monoxide. Archives of Microbiology Volume 161, Number 4 (1994), 345-351.

In an alternative preferred embodiment of the method according to the invention, ethyl acetate is formed in method step C) and an acetogenic bacterium is used.

Guidance for carrying out method step C) of this alternative preferred embodiment is described in WO2012162321.

In an alternative preferred embodiment of the method according to the invention, butanol is formed in method step C) and an acetogenic bacterium is used.

Guidance for carrying out method step C) of this alternative preferred embodiment is described in U.S.20110236941.

In an alternative preferred embodiment of the method according to the invention, hexanol is formed in method step C) and an acetogenic bacterium is used.

Guidance for carrying out method step C) of this alternative preferred embodiment is described in U.S.20100151543.

In an alternative preferred embodiment of the method according to the invention, 2,3-butanediol is formed in method step C) and an acetogenic bacterium is used.

Guidance for carrying out method step C) of this alternative preferred embodiment is described in U.S.20120252082 and WO2012131627.

In an alternative preferred embodiment of the method according to the invention, isopropanol is formed in method step C) and an acetogenic bacterium is used.

Guidance for carrying out method step C) of this alternative preferred embodiment is described in U.S.20120252083.

In an alternative preferred embodiment of the method according to the invention, 2-hydroxybutyric acid is formed in method step C) and an acetogenic bacterium is used.

Guidance for carrying out method step C) of this alternative preferred embodiment is described in EP12173010.

Method step C) is carried out using acetogenic bacteria, preferably under anaerobic conditions.

However, it is also possible and is therefore a preferred alternative embodiment of the method according to the invention, if method step C) is carried out under aerobic conditions.

This is to be understood as meaning that O₂ is present during method step C).

In the context of method step C), oxygen can be fed into the fermenter, for example, by introducing air.

In this context, preference is given to using a hydrogen-oxidizing bacterium in method step C).

The use of hydrogen-oxidizing bacteria likewise has the technical effect that the gas stream for method step C) can be reduced temporarily to a minimum and may even be completely interrupted. This type of bacteria, that in nature are used to surviving under the most adverse conditions, can remain in the fermenter for a long time without particular care and nutrition. In addition, the use of hydrogen-oxidizing bacteria causes the technical effect that excess energy formed can be utilized immediately since the bacteria, on reinstating the gas stream, immediately restart their metabolism and convert the gas to organic substance.

The term “hydrogen-oxidizing bacterium” is to be understood to mean a bacterium which is capable of chemolithoautotrophic growth and able to construct carbon skeletons having more than one carbon atom from H₂ and CO₂ in the presence of oxygen, in which the hydrogen is oxidized and the oxygen is used as terminal electron acceptor. According to the invention, it is possible to use either those bacteria which are naturally hydrogen-oxidizing bacteria or else bacteria which have become hydrogen-oxidizing bacteria by genetic modification, such as, for example, an E. coli cell which, as a result of recombinant insertion of the necessary enzymes, has been enabled to construct carbon skeletons having more than one carbon atom from H₂ and CO₂ in the presence of oxygen, in which the hydrogen is oxidized and the oxygen is used as terminal electron acceptor. Preferably, the hydrogen-oxidizing bacteria used in the method according to the invention are those which are already hydrogen-oxidizing bacteria as the wild type.

Hydrogen-oxidizing bacteria preferably used according to the invention are selected from the genera Achromobacter, Acidithiobacillus, Acidovorax, Alcaligenes, Anabena, Aquifex, Arthrobacter, Azospirillum, Bacillus, Bradyrhizobium, Cupriavidus, Derxia, Helicobacter, Herbaspirillum, Hydrogenobacter, Hydrogenobaculum, Hydrogenophaga, Hydrogenophilus, Hydrogenothermus, Hydrogenovibrio, ldeonella sp. O1, Kyrpidia, Metallosphaera, Methanobrevibacter, Myobacterium, Nocardia, Oligotropha, Paracoccus, Pelomonas, Polaromonas, Pseudomonas, Pseudonocardia, Rhizobium, Rhodococcus, Rhodopseudomonas, Rhodospirillum, Streptomyces, Thiocapsa, Treponema, Variovorax, Xanthobacter, Wautersia, wherein Cupriavidus is particularly preferred,
particularly from the species Cupriavidus necator (alias Ralstonia eutropha, Wautersia eutropha, Alcaligenes eutrophus, Hydrogenomonas eutropha), Achromobacter ruhlandii, Acidithiobacillus ferrooxidans, Acidovorax facili.s, Alcaligenes hydrogenophilus, Alcaligenes latus, Anabena cylindrica, Anabena oscillaroides, Anabena sp., Anabena spiroides, Aquifex aeolicus, Aquifex pyrophilus, Arthrobacter strain 11X, Bacillus schlegelii, Bradyrhizobium japonicum, Cupriavidus necator, Derxia gummosa, Escherichia coli, Heliobacter pylori, Herbaspirillum autotrophicum, Hydrogenobacter hydrogenophilus, Hydrogenobacter thermophilus, Hydrogenobaculum acidophilum, Hydrogenophaga flava, Hydrogenophaga palleronii, Hydrogenophaga pseudoflava, Hydrogenophaga taeniospiralis, Hydrogeneophilus thermoluteolus, Hydrogenothermus marinus, Hydrogenovibrio marinus, Ideonella sp. O-1, Kyrpidia tusciae, Metallosphaera sedula, Methanobrevibactercuticularis, Mycobacterium gordonae, Nocardia autotrophica, Oligotropha carboxidivorans, Paracoccus denitrificans, Pelomonas saccharophila, Polaromonas hydrogenivorans, Pseudomonas hydrogenovora, Pseudomonas thermophile, Rhizobium japonicum, Rhodococcus opacus, Rhodopseudomonas palustris, Seliberia carboxydohydrogena, Streptomyces thermoautotrophicus, Thiocapsa roseopersicina, Treponema primitia, Variovorax paradoxus, Xanthobacter autrophicus, Xanthobacter flavus, particularly from the strains Cupriavidus necator H16, Cupriavidus necator H1 or Cupriavidus necator Z-1.

In an alternative preferred embodiment of the method according to the invention, 2-hydroxybutyric acid is formed in method step C) and a hydrogen-oxidizing bacterium is used.

Guidance for carrying out method step C) of this alternative preferred embodiment is described in EP12173010.

In an alternative preferred embodiment of the method according to the invention, 1-butanol is formed in method step C) and a hydrogen-oxidizing bacterium is used.

Guidance for carrying out method step C) of this alternative preferred embodiment is described in EP13172030.2.

In an alternative preferred embodiment of the method according to the invention, propan-2-ol is formed in method step C) and a hydrogen-oxidizing bacterium is used.

Guidance for carrying out method step C) of this alternative preferred embodiment is described in EP13172030.2.

In an alternative preferred embodiment of the method according to the invention, acetone is formed in method step C) and a hydrogen-oxidizing bacterium is used.

Guidance for carrying out method step C) of this alternative preferred embodiment is described in EP13172030.2.

In an alternative preferred embodiment of the method according to the invention, 1-propene is formed in method step C) and a hydrogen-oxidizing bacterium is used.

Guidance for carrying out method step C) of this alternative preferred embodiment is described in EP13172030.2.

In an alternative preferred embodiment of the method according to the invention, butene is formed in method step C) and a hydrogen-oxidizing bacterium is used.

Guidance for carrying out method step C) of this alternative preferred embodiment is described in EP13172030.2.

The present invention further relates to an apparatus for carrying out the method according to the invention comprising:
a) a gas source for continuously providing a gas stream containing CO and/or CO₂;
b) a power-generating device for converting gases originating from the gas source into electrical energy;
c) a fermenter for converting gases originating from the gas source into at least one organic substance;
d) and means for selectively feeding the gas stream from the gas source to the power-generating plant and/or to the fermenter.

The apparatus according to the invention is preferably characterized in that the gas source is a blast furnace used in steel-making which continuously provides blast furnace gas as gas stream.

The power-generating device of the apparatus according to the invention preferably includes one generator driven by at least one turbine.

In this context, the turbine is preferably a gas turbine which can be operated entirely by the gas stream or by mixing with other fuels.

In accordance with the invention, the power-generating device preferably comprises a boiler for generating steam fired by the gas stream alone or by mixing with other fuels, and the turbine is a steam turbine which can be operated with the steam from the boiler.

The apparatus according to the invention is preferably characterized in that the fermenter hosts acetogenic bacteria and/or hydrogen-oxidizing bacteria.

The apparatus according to the invention is preferably characterized in that the means for selectively feeding the gas stream to the power-generating device and/or to the fermenter include lines and control elements connecting these apparatus.

In this context, it is preferable that the control elements and the lines are adapted to pressurize the fermenter and the power-generating device with the gas stream in a parallel and/or serial and/or individual manner.

With particular preference, the apparatus according to the invention is characterized in that all the components of the apparatus are integrated in one Verbund site.

The present invention further relates to the use of the apparatus according to the invention for carrying out the method according to the invention.

The use according to the invention is preferably for generating electrical energy and/or for producing at least one organic substance.

### EXAMPLES

The present invention will now be explained in more detail on the basis of a working example. Shown here:

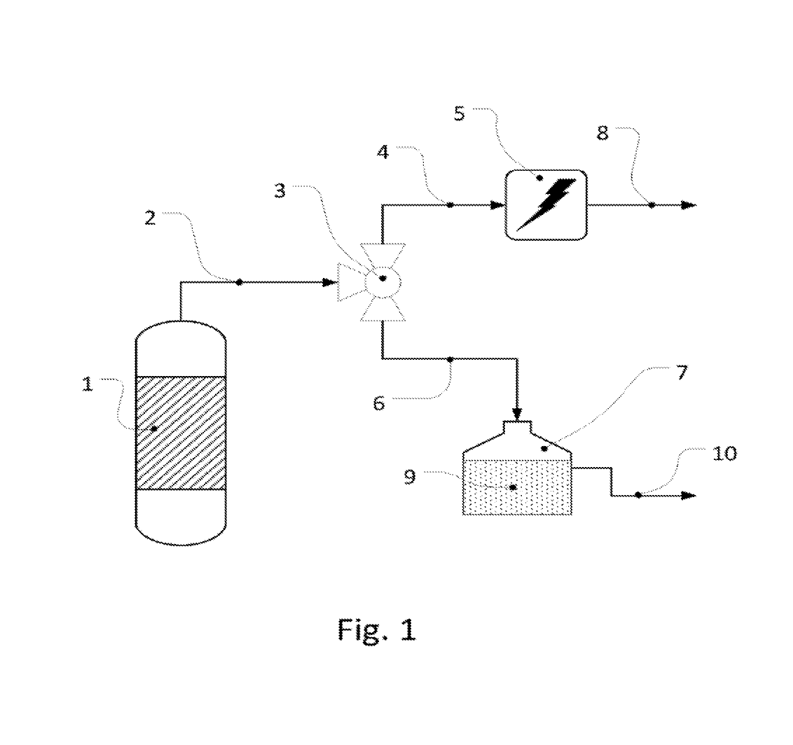

FIG. 1: Inventive apparatus for carrying out the method (schematic).

FIG. 1 shows the schematic construction of an apparatus according to the invention for carrying out the method.

A gas source 1, in the form of a conventional blast furnace for steel-making, supplies blast furnace gas continuously at its head, which is withdrawn via a corresponding gas line 2. The blast furnace gas produced during steel-making is a combustible coproduct gas having a nitrogen content of around 45-60% and a fraction of CO in the range of 20-30%. The blast furnace gas furthermore contains ca. 20-25% CO₂ and 2-4% H₂.

The blast furnace gas is passed to a control element 3. This is a valve known per se, which makes it possible to direct the inflowing gas from the gas source 1 either via a gas line 4 in the direction of a power-generating device 5 and/or via a gas line 6 in the direction of a fermenter 7. The control element 3 here enables the gas stream to be directed either completely and solely into the power-generating device 5 or completely and solely into the fermenter 7. In addition, the control element 3 may be set at intermediate positions which enable simultaneous supply of the gas stream to the power-generating device 5 and the fermenter 7 made up of identical or different proportions.

The gas passed into the power-generating device 5 is converted therein into electrical energy, by means of a conventional gas turbine or steam turbine process known per se, which is drawn off as electrical current 8 from the power-generating device 5. If a steam turbine is used, this may be operated exclusively with the gas from the gas source 1 or by adding external fuels. If a steam turbine process is used, the boiler for generating steam is also heated either with the gas from the gas source 1 or in addition with the aid of external fuels. It is also possible within the power-generating device 5 to couple a gas turbine process with a steam turbine process. The technologies described here for power generation from gas are well known from the prior art and need no further description here.

The proportions of the gas originating from the gas source 1, which are passed via the gas line 6 in the direction of the fermenter 7, are converted therein by bacteria 9 into an organic substance 10 which is drawn off from the fermenter 7.

The bacteria 9 preferably take the form of acetogenic bacteria or hydrogen-oxidizing bacteria. Suitable bacteria and fermentation processes for converting gases containing CO and/or CO₂ into organic substances are well known from the prior art cited above and therefore do not need to be described in detail.

Preparation example 1: 3-hydroxybutyric acid (3HB) using Cupriavidus necator cells with a gas stream comprising H₂ and CO₂ with interrupted gas supply.

A production phase of Cupriavidus necator PHB-4 was used for the biotransformation of oxyhydrogen to 3-hydroxybutyric acid (3HB). In this approach, the bacterium takes up H₂ and CO₂ from the conducted gas phase and forms 3HB. For the culturing, pressure-resistant glass bottles which can be sealed in an air-tight manner using a butyl rubber stopper were used.

To investigate the formation of 3-hydroxybutyric acid, the C. necator strain was firstly spread out on an LB-R agar plate containing antibiotic and incubated at 30° C. for 3 days.

For the purposes of the preculture, the strain was cultured in 200 ml of H16 mineral medium (modified according to Schlegel et aI.,1961) in pressure-resistant 500 ml glass bottles. The medium consisted of Na₂HPO₄×12 H₂O (9.0 g/l); KH₂PO₄ (1.5 g/l); NH₄Cl (1.0 g/l); MgSO₄×7 H₂O (0.2 g/l); FeCl₃×6 H₂O (10 mg/l); CaCl₂×2 H₂O (0.02 g/l); trace element solution SL-6 (Pfennig, 1974) (1 ml/l).

The trace element solution was composed of ZnSO₄×7 H₂O (100 mg/l), MnCl₂×4 H₂O (30 mg/l), H₃BO₃ (300 mg/l), CoCl₂×6 H₂O (200 mg/l), CuCl₂×2 H₂O (10 mg/l), NiCl₂×6 H₂O (20 mg/l), Na₂Mo₄×2 H₂O (30 mg/l). The pH of the medium was adjusted to 6.8 by addition of 1 M NaOH.

The bottle was inoculated with a single colony from the incubated agar plates and the culturing was carried out chemolithoautotrophically on an N₂/H₂/O₂/CO₂ mixture (ratio 80%/10%/4%/6%). The culture was incubated in an open water bath shaker at 28° C., 150 rpm and a gas flow rate of 1 l/h for 137 h, up to an OD>1.0. Gas was introduced into the medium via a gas supply frit which had a pore size of 10 μm and which was attached to a gas supply tube in the center of the reactor. The cells were subsequently centrifuged, washed with 10 ml of wash buffer (0.769 g/L NaOH, gassed through for at least 1 h at 28° C. and 150 rpm with a gas containing 6% CO₂) and recentrifuged.

For the production phase, sufficient washed cells were transferred from the growth culture to 200 mL of production buffer (NaOH (0.769 g/l), gassed through for at least 1 h at 28° C. and 150 rpm with a gas containing 6% CO₂, setting a pH of about 7.4) to set an OD600 nm of 1.0. The main culture was carried out chemolithoautotrophically in pressure-resistant 500 ml glass bottles at 28° C. and 150 rpm in an open water bath shaker with a gas flow rate of 1 l/h with an N₂/H₂/O₂/CO₂ mixture (ratio 80%/10%/4%/6%) for 188 h. The gas was introduced into the medium via a gas supply frit which had a pore size of 10 μm and which was attached to a gas supply tube in the center of the reactors. After a culturing period of 116 h, the gas supply was switched to 100% N₂ for 24 h, and then run again for a further 44 h with the original gas mixture (N₂/H₂/O₂/CO₂, ratio 80%/10%/4%/6%).

Sampling entailed removal of 5 ml samples in each case for the determination of OD600 nm, pH and the product spectrum. The product concentration was determined by semiquantitative 1H-NMR spectroscopy. The internal quantification standard used was sodium trimethylsilylpropionate (T(M)SP).

Over the culture period in the production phase, 270 mg/l 3HB formed after 116 h in the C. necator strain, whereas in the latter 48 h of the culture period after the gas supply interruption a further 140 mg/l 3HB was formed.

Preparation example 2: Ethanol and acetate using C. Ijungdahlii cells with a gas stream comprising H₂ and CO₂ with varied periods of interrupted gas supply.

In the following example, the wild type strain Clostridium Ijungdahlii is cultured autotrophically.

A complex medium was used consisting of 1 g/l NH₄Cl, 0.1 g/l KCl, 0.2 g/l MgSO₄×7 H₂O, 0.8 g/l NaCl, 0.1 g/l KH₂PO₄, 20 mg/l CaCl₂×2 H₂O, 20 g/l MES, 1 g/l yeast extract, 0.4 g/L L-cysteine HCl, 20 mg/l nitrilotriacetic acid, 10 mg/l MnSO₄×H₂O, 8 mg/l (NH₄)₂Fe(SO₄)₂×6 H₂O, 2 mg/l CoCl₂×6 H₂O, 2 mg/l ZnSO₄×7 H₂O, 0.2 mg/l CuCl₂×2 H₂O, 0.2 mg/l Na₂MoO₄×2 H₂O, 0.2 mg/l NiCl₂×6 H₂O, 0.2 mg/l Na₂SeO₄, 0.2 mg/l Na₂WO₄×2 H₂O, 20 μg/l d-biotin, 20 μg/l folic acid, 100 μg/l pyridoxine HCl, 50 μg/l thiamine HCl×H₂O. 50 μg/l riboflavin, 50 μg/l nicotinic acid, 50 μg/l Ca pantothenate, 1 μg/l vitamin B12, 50 μg/l p-aminobenzoate, 50 μg/l lipoic acid. Identical autotrophic cultures were carried out in 500 mL septum bottles with 100 mL of medium at a shaker frequency of 150 min⁻¹ in an open Innova 3100 water bath shaker from New Brunswick Scientific. The cultures were carried out at 37° C. without pH control. The reactors were pressurized with a synthesis gas mixture of 67% H₂ and 33% CO₂ at a positive pressure of 0.8 bar. To replenish spent gas, the gas atmosphere was completely replaced once a day in the headspace of the reactors and repressurized to 0.8 bar. Reactor 1 was pressurized with synthesis gas at the start and reactors 2, 3 and 4 after 24 h, 48 h and 72 h respectively. For the period without synthesis gas, the reactors were instead pressurized with a gas mixture of 67% N₂, 33% CO₂, also at 0.8 bar positive pressure.

At the start of the experiment, the reactor having an initial OD of 0.1 was inoculated with autotrophically grown cells. The preculture was performed continuously in a 1 L bottle with 500 mL of the abovementioned medium. The gas was supplied continuously to the culture using synthesis gas (67% H₂, 33% CO₂) at a volume flow rate of 3 L/h via a gas supply frit with a pore size of 10 μm. The cells were centrifuged off anaerobically in the late logarithmic growth phase at an OD of 0.64 (4500 rpm, 4300 g, 20° C., 10 min). The supernatant was discarded and the cell pellet resuspended in 10 mL of the abovementioned medium. The cells prepared were then used to inoculate the actual experiments.

Sampling entailed removal of 5 ml samples in each case for the determination of OD₆₀₀, pH and the product spectrum. The product concentration was determined by quantitative ¹H-NMR spectroscopy. The internal quantification standard used was sodium trimethylsilylpropionate (T(M)SP).

In reference experiment 1, gas consumption from the start (expressed by pressure drop Δp), a continuous decrease in pH, an increase in OD and an increase of the products acetate and ethanol was observed.

In reactors 2, 3 and 4 in the period without synthesis gas, no decrease in pH, no increase in OD and only a marginal increase in acetate could be observed in each case. If the gas atmosphere in the headspace of reactors 2, 3 and 4 is exchanged from N₂/CO₂ to H₂/CO₂ at the 24 h, 48 h and 72 h time points, and the cells thus supplied with synthesis gas, gas consumption, a decrease in pH, an increase in OD and an increase in the products acetate and ethanol in the further course of the experiment is surprisingly observed once again in each case.

| | | | | | NMR | |
|---|---|---|---|---|---|---|
| | Time, | Δp, | | | Ethanol, | Acetate, |
| Experiment | h | bar | pH | OD600 | mg/L | mg/L |
| 1 | 0.0 | — | 5.84 | 0.11 | 0 | 10 |
| MD-14-COx-034-K1 | 27.7 | 1.2 | 5.11 | 0.56 | 180 | 2275 |
| Gas supply was | 50.7 | 1.4 | 4.52 | 0.78 | — | — |
| initiated at timepoint | 78.2 | 1.4 | 4.56 | 0.95 | 450 | 7636 |
| t = 0 h | 144.2 | 0.4 | 4.49 | 0.87 | 528 | 10172 |
| (Reference experiment) | | | | | | |
| 2 | 0.0 | — | 5.82 | 0.12 | 0 | 9 |
| MD-14-COx-034-K2 | 27.7 | 0 | 5.79 | 0.11 | 0 | 89 |
| Gas supply was | 50.7 | 0.4 | 5.50 | 0.23 | — | — |
| initiated at timepoint | 78.2 | 1.3 | 4.81 | 0.69 | 229 | 4291 |
| t = 24 h | 144.2 | 1.6 | 4.35 | 0.85 | 237 | 5455 |
| 3 | 0.0 | — | 5.82 | 0.11 | 0 | 10 |
| MD-14-COx-034-K3 | 27.7 | 0 | 5.79 | 0.11 | — | — |
| Gas supply was | 50.7 | 0 | 5.77 | 0.10 | — | — |
| initiated at timepoint | 78.2 | 0.2 | 5.68 | 0.13 | 6 | 504 |
| t = 48 h | 144.2 | 1.6 | 4.77 | 0.52 | 219 | 2732 |
| 4 | 0.0 | — | 5.83 | 0.12 | 0 | 10 |
| MD-14-COx-034-K4 | 27.7 | 0 | 5.79 | 0.12 | — | — |
| Gas supply was | 50.7 | 0 | 5.73 | 0.10 | — | — |
| initiated at timepoint | 78.2 | 0.0 | 5.82 | 0.09 | 0 | 102 |
| t = 72 | 144.2 | 1.2 | 5.00 | 0.51 | 183 | 3120 |

Preparation example 3: Ethanol and acetate using Morella thermoautotrophica cells with a gas stream comprising H₂ and CO₂ with interrupted gas supply.

For the biotransformation of synthesis gas to acetate and ethanol, a culture with the Morella thermoautotrophica bacterium was carried out. In this approach, the bacterium takes up H₂ and CO₂ from the gas phase and uses these for cell growth and for the formation of acetate and ethanol. For the culturing, pressure-resistant glass bottles which can be sealed in an air-tight manner using a butyl rubber stopper were used. All culturing steps in which Morella thermoautotrophica cells were involved were carried out under anaerobic conditions.

For the cell culture of M. thermoautotrophica, each 2 mL cryoculture was anaerobically reactivated in 2×200 mL of medium (ATCC1754 medium: pH 6.0; 20 g/L MES; 1 g/L yeast extract, 0.8 g/L NaCl; 1 g/L NH₄Cl; 0.1 g/L KCl; 0.1 g/L KH₂PO₄; 0.2 g/L MgSO₄×7 H₂O; 0.02 g/L CaCl₂×2 H₂O; 20 mg/L nitrilotriacetic acid; 10 mg/L MnSO₄×H₂O; 8 mg/L (NH₄)₂Fe(SO₄)₂×6 H₂O; 2 mg/L CoCl₂×6 H₂O; 2 mg/L ZnSO₄×7 H₂O; 0.2 mg/L CuCl₂×2 H₂O; 0.2 mg/L Na₂MoO₄×2 H₂O; 0.2 mg/L NiCl₂×6 H₂O; 0.2 mg/L Na₂SeO₄; 0.2 mg/L Na₂WO₄×2 H₂O; 20 μg/L d-biotin; 20 μg/L folic aid; 100 μg/L pyridoxine HCl; 50 μg/L thiamine HCl×H₂O; 50 μg/L riboflavin; 50 μg/L nicotinic acid, 50 μg/L Ca pantothenate; 1 μg/L vitamin B₁₂; 50 μg/L p-aminobenzoate; 50 μg/L lipoic acid, ca. 67.5 mg/L NaOH; 100 mg/L L-cysteine hydrochloride). The culture was performed in a pressure-resistant 1000 ml glass bottle, which had been blanketed with a premixed gas mixture composed of 67% H₂, 33% CO₂ up to a positive pressure of 1 bar, at 58° C. and 150 rpm for 39 h in an open water bath shaker. The cells were cultured up to an OD of >0.2, centrifuged off and resuspended in fresh ATCC1754 medium.

For the culture phase, sufficient cells from the growth culture were transferred into 4×100 mL ATCC1754 medium to establish an OD₆₀₀ₙₘ of 0.1 in each case. The culture was performed in a pressure-resistant 500 ml glass bottle, which had been blanketed with gas up to a positive pressure of 1 bar, at 58° C. and 150 rpm for 91 h in an open water bath shaker. One culture in this case was blanketed at the start with a premixed gas mixture composed of 67% H₂, 33% CO₂, while the other three cultures were blanketed with 100% N₂. In one of the cultures blanketed with 100% N₂, the gas phase was exchanged after 24 h with the premixed gas mixture composed of 67% H₂, 33% CO₂, in one other after 48 h. The gas phase of the third culture blanketed with 100% N₂ did not have the gas phase exchanged.

Sampling entailed removal of 5 ml samples in each case for the determination of OD₆₀₀ₙₘ, pH and the product spectrum. The product concentration was determined by semiquantitative ¹H-NMR spectroscopy. The internal quantification standard used was sodium trimethylsilylpropionate (T(M)SP).

Over the culturing period, all cultures blanketed with 67% H₂, 33% CO₂ showed an increase in acetate from below 17 mg/L to above 2000 mg/L and in ethanol from below 8 mg/L to over 14 mg/L within 24 h under this gas atmosphere, even if these cultures had previously been cultured for 24 or 48 h under a 100% N₂ atmosphere.

### LIST OF REFERENCE NUMERALS

1 Gas source/blast furnace

2 Gas line for blast furnace gas

3 Control element

4 Gas line in the direction of the power-generating device

5 Power-generating device

6 Gas line in the direction of the fermenter

7 Fermenter

8 Electrical current

9 Bacteria

10 Organic substance

1. A method for utilizing a gas comprising CO and/or CO₂, the method comprising:
A) providing a gas stream comprising the gas comprising CO and/or CO₂,
B) converting at least a part of the gas stream into electrical energy,
C) converting at least a part of the gas stream to at least one organic substance in a biotechnological fermentation process, and
D) optionally repeating B) and C).

2. (canceled)

3. The method of claim 1, wherein B) is not carried out while C) is carried out.

4. The method of claim 3, wherein D) is carried out.

5. (canceled)

6. The method of claim 1, wherein A) comprises providing the gas stream from a gas source comprising a blast furnace gas from a blast furnace in steel-making.

7. (canceled)

8. The method of claim 1 B) comprises generating the electrical energy by means of a gas turbine process and/or a steam turbine process.

9. The method of claim 1, wherein the at least one organic substance C) comprises at least three carbon atoms.

10. (canceled)

11. The method of claim 1, wherein C) comprises fermenting at least a part of the gas stream with acetogenic bacteria.

12. (canceled)

13. The method of claim 1, wherein O₂ is added and a hydrogen-oxidizing bacterium is used C).

14. (canceled)

15. An apparatus for carrying out the method of claim 1, comprising:
a) a gas source for continuously providing a gas stream containing CO and/or CO₂;
b) a power-generating device for converting gases originating from the gas source into electrical energy;
c) a fermenter for converting gases originating from the gas source into at least one organic substance; and
d) means for selectively feeding the gas stream from the gas source to the power-generating plant and/or to the fermenter.

16. (canceled)

17. The apparatus of claim 15, the power-generating comprises a generator driven by at least one turbine.

18-19. (canceled)

20. The apparatus of claim 15, wherein the fermenter hosts acetogenic bacteria and/or hydrogen-oxidizing bacteria.

21. The apparatus of claim 15, the means for selectively feeding the gas stream to the power-generating device and/or to the fermenter comprises lines and control elements connecting these apparatus.

22. (canceled)

23. The apparatus of claim 15, wherein all components of the apparatus are integrated in one Verbund site.

24. The method of claim 1, being carried out with an apparatus comprising:
a) a gas source for continuously providing a gas stream containing CO and/or CO₂;
b) a power-generating device for converting gases originating from the gas source into electrical energy;
c) a fermenter for converting gases originating from the gas source into at least one organic substance; and
d) means for selectively feeding the gas stream from the gas source to the power-generating plant and/or to the fermenter.

25. The method of claim 24, wherein the method generates electrical energy and/or produces at least one organic substance.
